SEQUENCE LISTING (2 sequences)

SEQ ID NO: 1 - DNA, 57 bp, Artificial
  Primer for PCR (forward); V3 region of 16S rRNA
cgcccgccgcgcgcggcgggcggggcgggggcacggggggcctacgggaggcagcag

SEQ ID NO: 2 - DNA, 17 bp, Artificial
  Primer for PCR (reverse); V3 region of 16S rRNA
attaccgcggctgctgg

(19) United States
(12) Patent Application Publication
(10) Pub. No.: US 2009/0294287 A1
(43) Pub. Date: Dec. 3, 2009

(54) MICROCHIP ELECTROPHORESIS METHOD AND DEVICE

(75) Inventors: Tomoyuki Morita, Kanagawa (JP); Takashi Matsumura, Kanagawa (JP); Akiko Miya, Kanagawa (JP); Hiroyuki Yamada, Kanagawa (JP)

Correspondence Address: BROWDY AND NEIMARK, P.L.L.C.;624 NINTH STREET, NW, SUITE 300, WASHINGTON, DC, 20001-5303, US

(73) Assignee: Ebara Corporation, Ohta-ku (JP)

(21) Appl. No.: 11/915,491

(22) PCT Filed: May 10, 2006
(86) PCT No.: PCT/JP2006/309774

§ 371 Date: Nov. 26, 2007

(30) Foreign Application Priority Data
May 24, 2005 (JP) 2005-150770

(51) Int. Cl. C07K 1/26

(52) U.S. Cl. 204/451; 204/601

(57) ABSTRACT

An separation method comprising a temperature control process useful for microchip electrophoresis such as microchip DGGE is provided along with a device therefor.

The present invention relates to a microchip electrophoresis method for separating double-stranded nucleic acids by means of differences in nucleotide sequence while maintaining a preset temperature, wherein the temperature during separation of double-stranded nucleic acids in an separation region comprising an separation microchannel is controlled to within ±2.5° C. of the preset temperature.

### BACKGROUND OF THE INVENTION

The present invention relates to a microchip electrophoresis method and device for separating double-stranded nucleic acids by means of differences in nucleotide sequence at a preset temperature while maintaining that preset temperature.

The following prior art is known for separation using capillaries or microchips. Japanese Patent Application 2001-515204 describes a microfluidic system equipped with a temperature-responsive energy source and a sensor connected functionally to a channel for determining the temperature of a fluid. Japanese Patent Application Laid-open No. 2003-117409 describes a device for microchemistry molded from heat-resistant plastic and provided with a temperature regulation means. Japanese Patent Application Laid-open No. 2004-279340 describes a microchip provided with a plurality of temperature sensors. International Patent Application WO2002/090912 describes a method for measuring the temperature of a liquid phase inside the microchannel of a microchip, wherein the temperature of a liquid phase inside the microchannel of a microchip is measured without contact by detecting the fluorescence intensity of fluorescent substances. However, these prior technologies do not relate to techniques for separating double-stranded nucleic acids by means of differences in nucleotide sequence.

Japanese Patent Application H10-502738 relates to a technique for separating double-stranded nucleic acids in a capillary. This pertains to a method of separating double-stranded nucleic acids by non-micelle electrophoresis in an electrophoretic separation medium, using the fact that the melting temperature varies with differences in the DNA nucleotide sequence. That is, this separation technique uses the temperature gradient over time rather than the concentration gradient of the denaturant as the condition for weakening the hydrogen bonds between the double strands.

The present application pertains to a microchip electrophoresis method for denaturing and separating double-stranded nucleic acids by means of the action of a denaturant at a preset temperature that is roughly stable in a process for separating double-stranded nucleic acids (hereunder sometimes called a “nucleic acid sample”). The inventors in this case first investigated what effect the gel temperature has on detection results in this kind of electrophoresis.

FIG. 11 shows the results for separation of double-stranded nucleic acids by constant denaturing gel electrophoresis (CDGE) at different preset temperatures. Lane 1 and Lane 2 are samples with different nucleotide sequences, while Lane 3 is a mixture of the samples of Lane 1 and Lane 2. While no separation at all occurred at 45° C., 52.5° C. or 55° C., there was some separation at 47.5° C. and complete separation at 50° C. Thus, it appears that temperature control with a precision of ±1° C. is necessary in constant denaturing gel electrophoresis. Even higher-precision temperature control is required when the difference in nucleotide sequence is smaller than in the case of these two samples.

In denaturing gradient gel electrophoresis (DGGE), the actual temperature control does not need to be as precise as in CDGE because resolution is better than with CDGE using the concentration gradient of the denaturant. Because the temperature dependence is similar, however, changes in temperature should have essentially the same effect on the reproducibility and detection efficiency (efficiency with which differences in nucleotide sequence are separated) of the DGGE detection results.

Thus, in microchip electrophoresis in which double-stranded nucleic acids are separated on the basic of differences in nucleotide sequence while maintaining any preset temperature that is optimal for separation, high reproducibility and detection efficiency cannot be obtained if the preset temperature is not controlled precisely.

However, there is another problem with temperature control in microchips. Temperature control is generally difficult because microchips have a low heat capacity. For example, they tend to respond sensitively to the temperature of the heater, or to reflect as is the temperature distribution of the heater. Since they also dissipate heat easily, this is actually an advantage of microchips because when it is necessary to raise or lower the temperature (as in PCR) such temperature changes can be accomplished rapidly. This property is inconvenient however when attempting to control with high precision a preset temperature which needs to be uniform throughout a microchip for separation. In particular, the glass or plastic used as materials in microchips are less thermally conductive than metal, making temperature control difficult. For example, local rises in temperature are likely.

Moreover, it is preferable that sample separation (that is, DNA denaturation) does not occur during the process of introducing a nucleic acid sample into an separation microchannel (specifically, the process of introduction from a sample introduction microchannel). If the nucleic acid sample is exposed to the DNA melting temperature as it is being introduced, DNA separation will be initiated inside the sample introduction microchannel, interfering with optimal introduction of a uniform nucleic acid sample. Even in the process of detecting the nucleic acid sample (or the detection region), detection sensitivity is lower at the melting temperature due to desorption of the dye used to stain the DNA.

However, various methods are being studied for temperature control using capillaries, as described in Patent Application H10-502738. The main subject of this application is a control method suited to forming a temperature gradient over time in a capillary. Because in general one independent capillary is used as the separation microchannel in capillary electrophoresis, it is easy to apply temperature control to the capillary as a whole by an external operation or the like in this configuration. Unlike a capillary, however, a microchip normally has a plurality of microchannels in one plate. For example, a DGGE microchip typically has a complex structure comprising a sample introduction microchannel and an separation microchannel with different functions intersecting each other on the same plate.

As explained above, because of unique problems stemming from material restrictions and the complex configuration of the channels and the like, suitable temperature control methods in microchip electrophoresis and microchip denaturing gradient gel electrophoresis (hereunder “microchip DGGE”) in particular need to be studied from a different perspective than in the case of capillaries.

### SUMMARY OF THE INVENTION

It is an object of the present invention to provide an separation method comprising a temperature control process useful for microchip electrophoresis such as microchip DGGE for example, along with a device therefor.

As a result of exhaustive research aimed at solving the aforementioned problems, the inventors discovered unexpectedly that in microchip electrophoresis high-precision temperature control within a range of ±2.5° C. or preferably ±1° C. of a preset temperature provides high reproducibility and detection efficiency, and perfected the present invention based on this novel finding.

That is, the present invention provides a microchip electrophoresis method for separating double-stranded nucleic acids by means of differences in nucleotide sequence while maintaining a preset temperature, wherein the temperature during the process of separating the double-stranded nucleic acids is controlled within ±2.5° C. of the preset temperature.

In this method, at least one temperature selected from the temperature during the process of introducing the double-stranded nucleic acids into an separation microchannel, the temperature during the process of separating the double-stranded nucleic acids in the separation microchannel and the temperature during the process of detecting the separated double-stranded nucleic acids may be controlled independently.

The present invention is a microchip electrophoresis device for separating double-stranded nucleic acids based on differences in nucleotide sequence while maintaining a preset temperature, the microchip electrophoresis device being provided with a temperature control device capable of controlling the temperature of the region of the aforementioned separation microchannel to within ±2.5° C. of the aforementioned preset temperature.

The device of the present invention may also be provided with a temperature control device capable of independently controlling at least one temperature selected from the temperature of the region of a sample introduction microchannel for introducing the double-stranded nucleic acids into the separation microchannel, the temperature of the region of the separation microchannel and the temperature of the region for detecting the separated double-stranded nucleic acids.

The device of the present invention may be provided with one or a plurality of temperature sensors on the microchip body. It also may be provided with a plurality of heaters.

From a different perspective, the inventors discovered that when used as the DNA separation medium in microchip electrophoresis, a specific hydroxyethylcellulose (HEC) provides resolution equal to or greater than the solid gels used in conventional DGGE. The present invention relates to the use of hydroxyethylcellulose, which is useful as an separation medium in microchip electrophoresis.

In FIG. 12, (a) through (d) show the amounts of DNA separated in hydroxyethylcellulose solution of differing concentrations. The results for molecular weight separation of DNA in conventional solid gel (polyacrylamide) are shown in (e). Moreover (f) shows the results when comparing the results (d) and (e) in terms of selectivity. Selectivity is an indicator of DNA separation ability of a DNA separation medium. The greater the selectivity, the greater the ability to separate DNA. In (f) selectivity is indicated relative to DNA size, with DNA size shown on the horizontal axis. As shown by these test results, the 1.5% HEC solution is superior to the conventional solid gel within the range of 75 to 300 bp, which includes the range around 200 bp targeted by DGGE. If the HEC concentration is below 1%, the DGGE chip will not have the necessary resolution and the retention time will be longer, while if the HEC concentration exceeds 2%, the operation of filling the microchip with the HEC solution becomes more difficult.

However, the optimal concentration of HEC depends greatly on the properties (average molecular weight, molecular weight distribution, etc.) of the HEC used, Based on the aforementioned experiments, it appears that an HEC concentration of 1.5% is desirable for hydroxyethylcellulose with a number-average molecular weight of 90,000 to 105,000 but this concentration could be varied in the range of 0.1 to 10% depending on the average molecular weight and molecular weight distribution. Further research has shown that in fact a 1.75% HEC solution is more desirable than a 1.5% HEC solution.

Based on these findings, the present invention relates to a microchip electrophoresis method for separating double-stranded nucleic acids by means of differences in nucleic acid sequence while maintaining a preset temperature, wherein the temperature during the process of separating the double-stranded nucleic acids is controlled within at least ±2.5° C. or preferably ±1° C. of the aforementioned preset temperature, and wherein hydroxyethylcellulose with a number-average molecular weight of 90,000 to 105,000 is used as the separation medium. Preferably a roughly 1.5% solution of the aforementioned hydroxyethylcellulose is used. More preferably, a roughly 1.75% solution of the hydroxyethylcellulose is used.

### BRIEF DESCRIPTION OF THE DRAWINGS

FIG. 1 is a diagram showing a DGGE microchip for spatial temperature control.

FIG. 2 is a rough diagram showing another embodiment of a DGGE microchip for spatial temperature control.

FIG. 3 is a flow chart of a DGGE microchip temperature control method for temperature control over time.

FIG. 4 is a cross-section of a microchip having a plurality of temperature sensors on the top.

FIG. 5 is a cross-section of a microchip having a plurality of temperature sensors on the inside.

FIG. 6 is a cross-section of a microchip having a plurality of temperature sensors at the bottom.

FIG. 7 is a cross-section of a microchip having a plurality of temperature sensors and a plurality of heaters.

FIG. 8 is a top view of a microchip having a plurality of temperature sensors and a plurality of heaters.

FIG. 9 is a top view of a DGGE microchip having temperature sensors and heaters in each region.

FIG. 10 is a cross-section of a DGGE microchip having temperature sensors and heaters in each region.

FIG. 11 is a photographic image showing the effect of temperature change on constant denaturing gel electrophoresis.

FIG. 12 is a graph showing the nucleic acid molecular weight separation effects of differing concentrations of hydroxyethylcellulose.

### DESCRIPTION OF THE PREFERRED EMBODIMENTS

This application relates to a microchip electrophoresis method for separating a nucleic acid sample by the action of a DNA denaturant at a preset temperature. One mode of the present method, microchip DGGE, is explained below as an example.

The principle of DGGE exploits a phenomenon in which when the charge of nucleic acid nucleotides is neutralized with a DNA denaturant such as urea or formamide, the hydrogen bonds between nucleotides are cleaved, dissociating the double-stranded nucleic acid into single-stranded nucleic acid. The nucleic acid sample may be double-stranded DNA which has been amplified by PCR after addition to one end of an artificial DNA sequence (GC clamp) which resists dissociation into single-stranded DNA even at a high DNA denaturant concentration. When a double-stranded nucleic acid with an attached GC clamp is electrophoresed in gel with a DNA denaturant concentration gradient, the end of the double-stranded nucleic acid without the GC clamp will be dissociated into single strands at a particularly denaturant concentration, and the migration speed of the nucleic acid molecule will fall. Because the denaturant concentration at which the double-stranded nucleic acid is denatured into single strands is dependent on its nucleotide sequence, double-stranded nucleic acid molecules with different nucleotide sequences will migrate different distances when electrophoresed on the same concentration gradient region. Double-stranded nucleic acids can thus be separated by means of differences in nucleotide sequence.

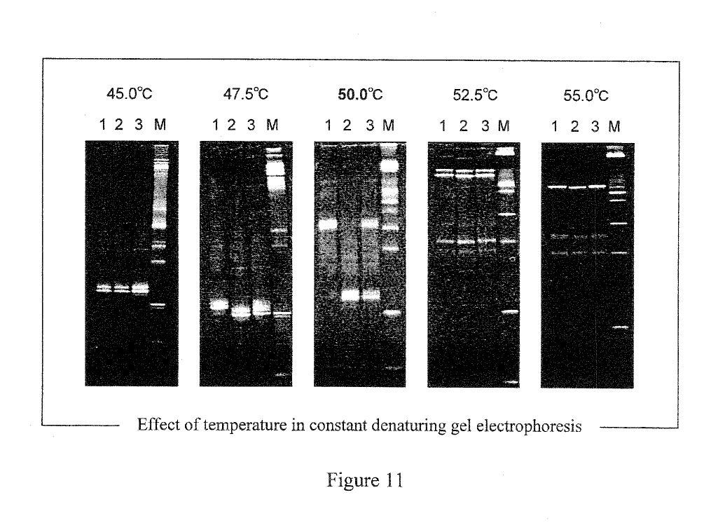

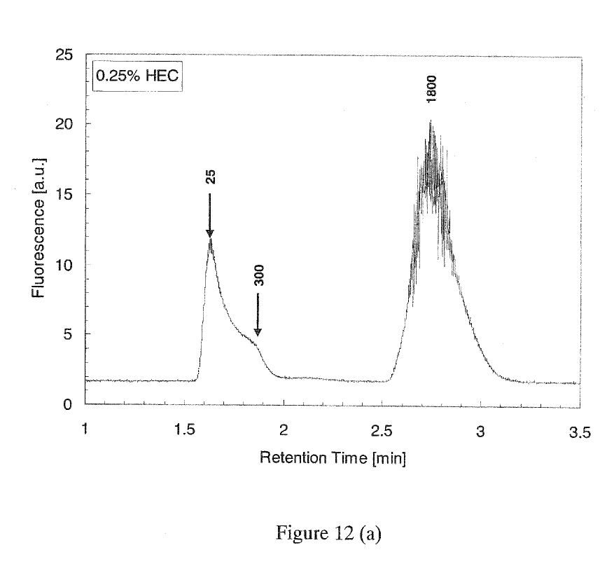

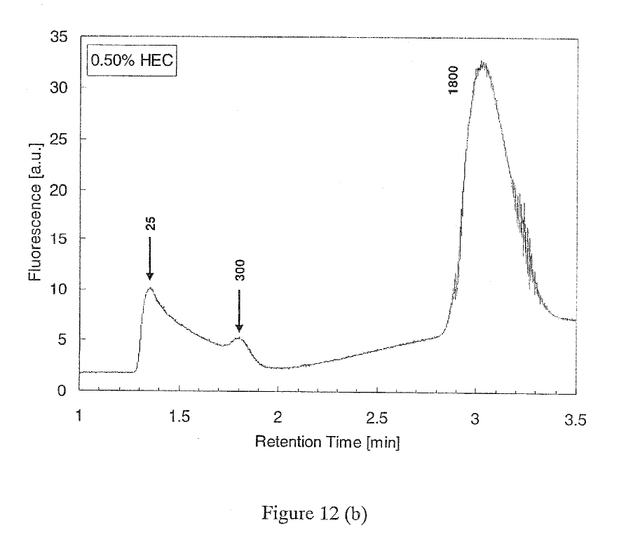

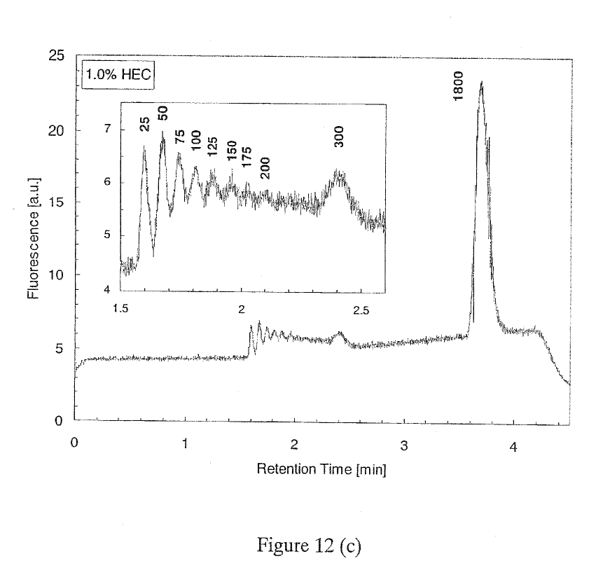

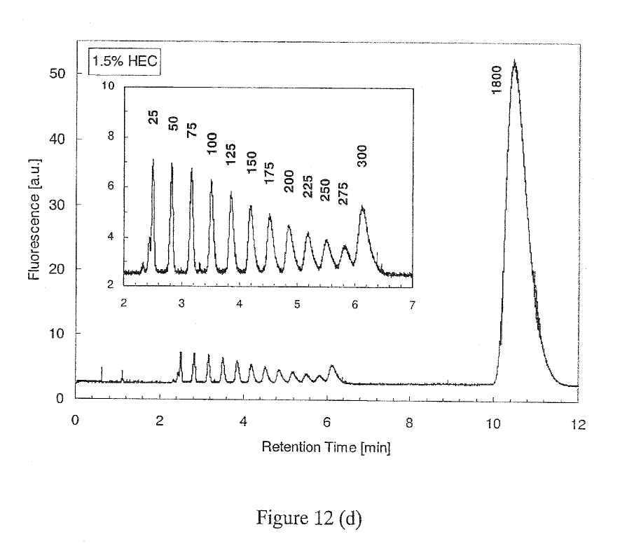

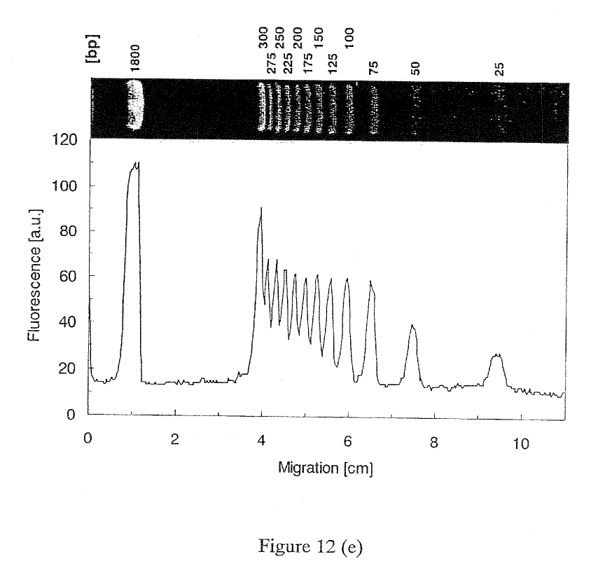

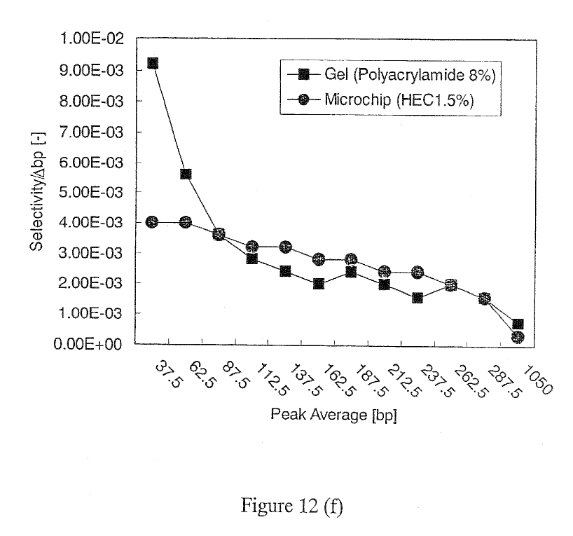

When separating double-stranded nucleic acids by means of differences in nucleotide sequence, the separation temperature greatly affects whether or not a nucleic acid sample is separated as well as the reproducibility of the migration difference. This is suggested by CDGE experiments performed by the inventors in this case (FIG. 11). In this sense, it is easy to maintain a uniform temperature distribution of the whole gel in conventional DGGE using solid gel because the gel as a whole is completely immersed in temperature-controlled buffer in the tank during electrophoresis. Consequently, there has previously been no particular need to investigate the temperature distribution of the gel or special control methods therefor.

Like normal-scale DGGE and the like, microchip DGGE is temperature dependent because separation is accomplished using differences in the degree of denaturing of double-stranded nucleic acids. Because of the high temperature responsiveness and difficulty of temperature control of microchips, this temperature dependence causes problems that cannot be ignored in microchip DGGE. A temperature distribution in the range of 5 to 6° C. often occurs in microchip temperature control, which is normally within the tolerance range for ordinary synthetic reactions, chemical reactions and the like. However, because microchip electrophoresis is highly temperature dependent it does not allow such temperature variation.

In the microchip electrophoresis method of the present invention, a preset temperature suitable for separating a nucleic acid sample is controlled so as to maintain a temperature in the range of within ±2.50° C. or preferably ±1° C. of the preset temperature in a process for separating a double-stranded nucleic acid sample according to the acting frequency of a denaturant which fluctuates depending on the migration distance, allowing the double-stranded nucleic acid to be properly separated. In microchip DGGE, it may be impossible to separate a normally separable nucleic acid sample if the temperature during separation of the nucleic acid sample is not controlled within the range of ±2.5° C. of the preset temperature. That is, the certainty of obtaining an optimum nucleic acid separation effect is greatly improved by controlling the temperature within the range of ±2.5° C. of the preset temperature. Moreover, by controlling it within the range of ±1° C. of the preset temperature it is possible not only to better assure separation, but also to improve the reproducibility of the nucleic acid sample detection times and to specify each double-stranded nucleic acid according to its respective detection time. This kind of high-precision temperature control is particularly desirable in the case of DNA samples with small differences in nucleotide sequence. Control in the range of ±1° C. of the preset temperature can be achieved by improving heat conductivity between microchip and heater. For example, a highly thermally conductive material can be inserted between microchip and heater. The material used can be any with high heat conductivity, and preferably is a material with a heat conductivity of 2×10⁻³ cal/cm·sec·° C. or more whereby heat conductivity is improved by eliminating the air layer and improving adhesion between microchip and heater. Examples of such materials include aluminum, copper and other metals and thermally conductive grease and the like.

In the present invention, “controlling within a prescribed range of the preset temperature” includes controlling to reduce spatial temperature variation relative to the preset temperature (that is, reducing the temperature distribution in the longitudinal direction of one region of the separation microchannel) and/or controlling to reduce temperature variation over time relative to the preset temperature (that is, maintaining a fixed temperature from the beginning to the end of the separation process). Specifically, it includes controlling to maintain the temperature of all areas of the separation microchannel within ±2.5° C. or preferably ±1° C. of the preset temperature, and/or controlling to maintain the temperature over time during the process of separating nucleic acids in the separation microchannel to within ±2.5° C. or preferably 1° C. of the preset temperature.

In the present invention, the “preset temperature” is a temperature believed to be optimal for obtaining proper separation results for a nucleic acid sample, and for practical purposes the preset temperature will differ depending not only on the properties of the separation target (nucleic acid length, GC content, etc.) but also on the properties of the separation medium used and the like. A person skilled in the art can discover the optimal preset temperature through preliminary experiments with the separation target and separation conditions. The “preset temperature” of the present invention is basically the temperature to be maintained during the process of separating nucleic acids.

In a preferred method of the present invention, the temperature of the process of introducing the nucleic acid sample into the separation microchannel (introduction process), the temperature of the process of separating the nucleic acid sample in the microchannel (separation process) and the temperature of the process of detecting the nucleic acid sample (detection process) are controlled independently.

When the temperatures of the introduction process and separation process are the same the nucleic acid also becomes separated in the sample introduction microchannel, so that it may not be possible to introduce the nucleic acid sample into the separation microchannel uniformly and without variation, or in other words at a fixed concentration. When nucleic acids are introduced after separation has started, the optimal nucleic acid concentration ratio (concentration ratio between separation bands), which should be seen after separation is complete, is not achieved. To solve this problem, in one mode of the present invention a relatively low temperature at which separation does not occur is set for the introduction process, allowing the nucleic acid sample to be introduced into the separation microchannel at the optimal concentration.

When the temperatures for the detection process and separation process are the same, the nucleic acid is detected at a high temperature at which the nucleic acid is separated. At high temperatures the fluorescent dye used for the nucleic acid is liable to desorption, reducing the level of detected fluorescence and detracting from detection sensitivity. To solve this problem, in one mode of the present invention the detection process temperature is controlled at a lower temperature than the separation process temperature, allowing tiny amounts of nucleic acid sample to be detected while maintaining detection sensitivity.

The microchip for implementing this method is provided with a temperature control device for controlling the temperature of the microchip. The temperature control device is made up of a temperature sensor and heater arranged in suitable locations on the microchip body such as along the separation microchannel and sample introduction microchannel, and a control device body connecting these. The control device body is made up of a general-use computer comprising a CPU or the like having a computing function. Upon receiving an input signal from the temperature sensor, the control device body outputs a signal which drives the heater based on the discrepancy between the preset target temperature and the measured temperature, thus accomplishing feedback control and the like. Preferably, a plurality of temperature sensors and a plurality of heaters are provided on the microchip body. The temperature sensors are preferably provided on the microchip body separately from the heaters.

The size of the microchip is generally a few cm by a few cm, with a thickness of a few mm. The heat capacity is therefore very low, and temperature control of the microchip is not easy because it is so sensitive to the temperature variation or temperature distribution of the heaters. Also, in general microchips are made of glass, quartz, plastic, silicon resin or the like, all of which have poorer thermal conductivity than metal, making temperature control difficult because of the likelihood of temperature distributions such as local temperature elevation in the microchips. When controlling the temperature of such a microchip with a small heat capacity and poor thermal conductivity, in a conventional temperature control device with a temperature sensor on the heater itself, there is likely to be a disparity between the microchip temperature and heater temperature, making highly precise temperature control impossible. One means of solving this problem is to arrange a plurality of temperature sensors on the microchip body at a specific distance from the respective heaters as in the present invention. In this way, the temperature of the microchip itself can be measured, allowing highly precise temperature control within ±1° C. of the preset temperature.

Fixing a plurality of temperature sensors on the microchip also allows the temperature of a target region on the microchip to be measured, so that the heaters can be controlled in accordance with the measured temperature at that target region. Thus, the temperature of a target region on a microchip which is liable to temperature distribution can be controlled reliably to within ±10° C. of the preset temperature.

Providing a plurality of heaters on the microchip also makes it easier to uniformly control the temperature distribution within specific regions of the microchip. At the same time, a plurality of regions of the same microchip can be controlled at different temperatures. In the case of microchip DGGE, this configuration is advantageous because the respective temperatures of the sample introduction microchannel, the separation microchannel and the region for detecting the separated nucleic acid sample can be easily controlled independently, and the optimal temperature can be obtained for each process.

Next, one embodiment of the present invention is further explained using drawings.

As explained above, in a preferred DGGE microchip of the present invention, the temperature of the process of introducing the nucleic acid sample into the separation microchannel (sample introduction process), the temperature of the process of separating the nucleic acid sample in the separation microchannel (separation process) and the temperature of the process of detecting the nucleic acid sample (detection process) are each controlled independently. Methods of controlling temperatures independently in this way include methods of variably controlling the temperature of the regions where each of the aforementioned processes is performed independently (spatial control), and methods of controlling the microchip overall over time as each process progresses (temporal control).

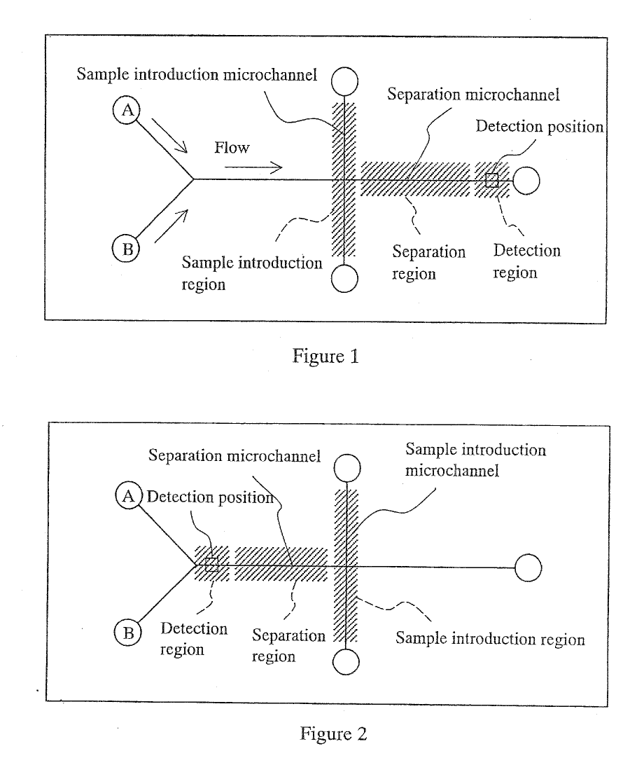

FIGS. 1 and 2 show an example of a DGGE microchip for spatial control. The denaturant concentration gradient is formed by mixing varying proportions of solutions A and B containing different concentrations of denaturant (typically buffer A containing no denaturant and buffer B containing a fixed concentration of denaturant, each of which may also contain a fixed concentration of a polymer separation medium), and moving them to the separation region comprising the separation microchannel. Once the denaturant concentration gradient has formed in the separation microchannel, the nucleic acid sample is introduced from the sample introduction microchannel into the intersection with the separation microchannel. The nucleic acid sample introduced into the intersection with the separation microchannel is electrophoresed in the denaturant gradient in the separation microchannel, moves in a specific direction and is separated. As it is separated, the nucleic acid sample is detected optically as it passes through a specific detection site on the separation microchannel.

The position of the separation region differs depending on the direction of migration of the nucleic acid sample in the separation microchannel. In the DGGE microchip of FIG. 1, the detection position is located to the right of the sample introduction microchannel in the figure. In the DGGE microchip of FIG. 2, the detection position is located to the left of the sample introduction microchannel in the figure.

The sample introduction region comprising the sample introduction microchannel is independently controlled at a temperature below that at which the nucleic acid sample is separated so that the nucleic acid sample can be introduced uniformly. The detection region comprising the detection position in the separation microchannel is also controlled independently at a temperature below that at which the nucleic acids are separated so as to prevent desorption of the nucleic acid dye and prevent a decrease in detection sensitivity. The separation region comprising the separation microchannel is controlled so as to maintain a temperature within ±2.5° C. of the preset temperature at which the nucleic acid sample is separated.

Typically, the temperature of the sample introduction region is 20 to 40° C. while the preset temperature of the separation region is 40 to 70° C. and the temperature of the detection region is 20 to 40° C., but these temperatures can be studied in advance and determined appropriately depending on the conditions of use such as the types and concentrations of the nucleic acid sample and denaturant and the like.

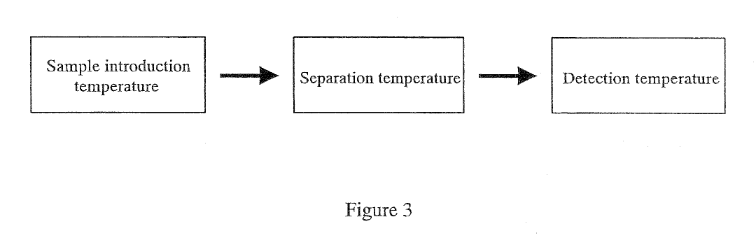

FIG. 3 shows the flow chart of a microchip temperature control method for achieving temporal control. When introducing the sample, the microchip as a whole is controlled at a temperature below that at which the nucleic acids are separated so as to introduce the nucleic acid sample uniformly. Next, during separation, the microchip as a whole is controlled at a temperature in the range of within ±2.5° C. of the preset temperature at which the nucleic acids are separated. Finally, during detection, the microchip as a whole is controlled at a temperature below that at which the nucleic acids are separated so as to inhibit desorption of the nucleic acid dye and prevent a reduction in detection sensitivity. The temperature during each operation is affected by the test conditions including the types of nucleic acid sample and concentrations of denaturant, but typically the temperature is about 20 to 40° C. during sample introduction, a preset temperature of 40 to 70° C. during separation and 20 to 40° C. during detection.

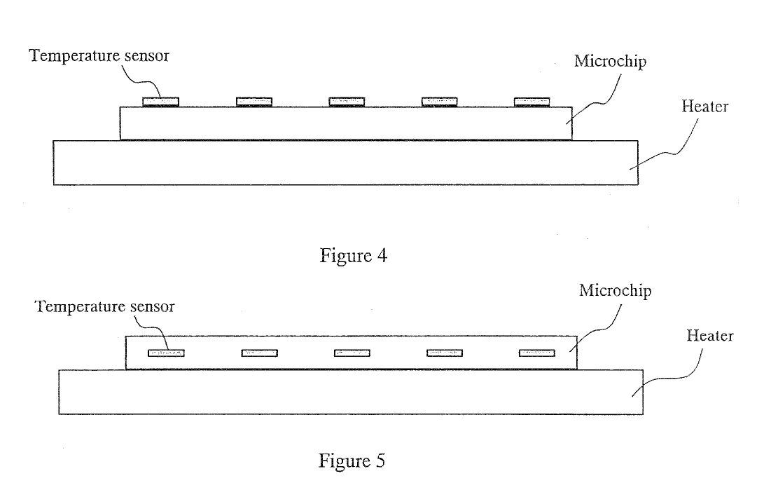

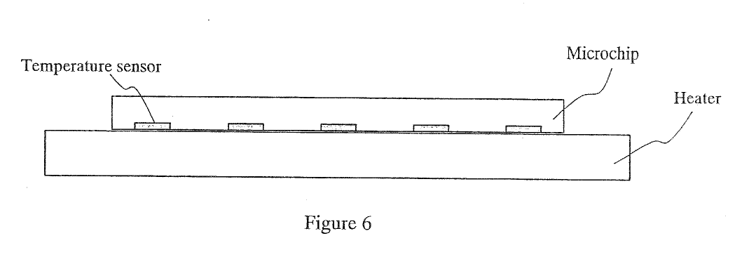

FIGS. 4, 5 and 6 show cross-sections of microchips provided with a plurality of temperature sensors. In the microchip of FIG. 4, the temperature sensors are fixed on the upper surface of the microchip, while in the microchip of FIG. 5 the temperature sensors are embedded inside the microchip and in the microchip of FIG. 6 the temperature sensors are incorporated into the lower surface of the microchip. Thus, the temperature sensors can be provided on the upper surface, the inside or the lower surface of the microchip.

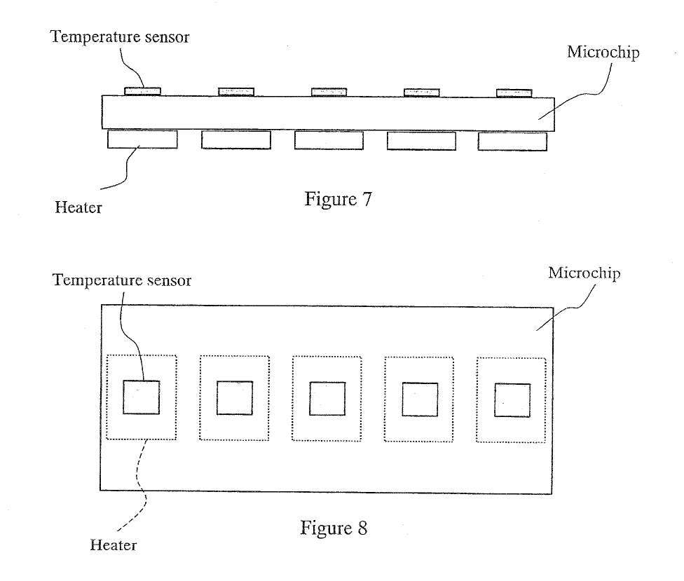

FIGS. 7 and 8 show one example of a microchip provided with a plurality of heaters and a plurality of temperature sensors. FIG. 7 is a cross section while FIG. 8 is a top view. In this microchip set, the temperature of each region is controlled by means of a temperature sensor located on the upper surface and a heaters located on the lower surface. With this arrangement the temperatures of different regions of the microchip can be controlled independently, and since the temperature sensors detect the temperature of a microchip that is being heated from the opposite side, the temperature control can reflect the actual temperature distribution of the microchip. This kind of control is useful for reducing temperature distribution when variation is likely in the temperature distribution of a microchip.

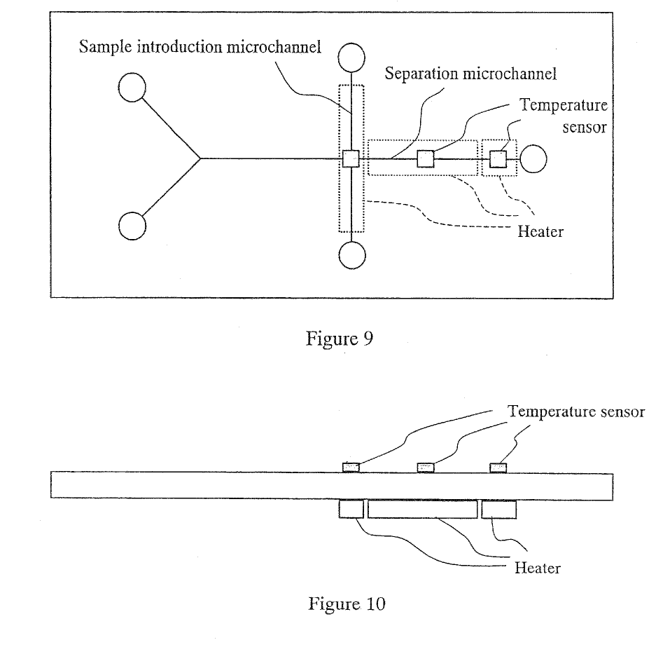

FIGS. 9 and 10 show one example of a DGGE microchip equipped with a temperature sensor and a heater in each region. FIG. 9 is a top view and FIG. 10 is a cross-section. The sample introduction region comprising the sample introduction microchannel, the separation region comprising the separation microchannel and the detection region comprising the detection position are each provided with a temperature sensor and a heater. With this arrangement the temperature of the sample introduction region, the temperature of the separation region and the temperature of the detection region can each be controlled independently. This microchip allows the uniform introduction of the nucleic acid sample at a low temperature which inhibits nucleic acid separation in the sample introduction process, as well as the highly sensitive detection of a nucleic acid sample at a low temperature which inhibits desorption of the fluorescent dye from the nucleic acid. The microchip of FIGS. 9 and 10 is provided with one temperature sensor and one heater in each region, but each region could also be provided with a plurality of temperature sensors and a plurality of heaters.

The microchip body to be used in the present invention can be manufactured by known photolithography techniques. As the method for moving the liquid in the microchip, an electroosmosis flow or pump suited to moving a small quantity of liquid can be used based on known methods. Electrophoresis can also be based on known methods using suitable electrodes and power sources.

The temperature sensors used in the present invention may be thermocouples, resistance thermometer sensors, thermistors or the like. Radiation thermometers may also be used when it is difficult to fix the temperature sensors depending on the dimensions of the microchip, the materials and other conditions. A thin film of temperature-dependent metal may also be patterned directly on the microchip body by plating, vapor deposition, sputtering, ion plating, pasting or the like to form the temperature sensors.

In addition to ordinary heaters, Peltier elements can also be used as heaters in the present invention. Both heating and cooling can be accomplished using Peltier elements, allowing a wide range of more precise temperature control with greater responsiveness. A thin film of a metal with high electrical resistance may also be patterned directly on the microchip body by plating, vapor deposition, sputtering, ion plating, pasting or the like to form the heaters. Alternatively a transparent conductive film such as indium tin oxide may be provided to form the heaters or heat exchangers can be used.

In the microchip of the present invention, an external fan can be used for cooling. The addition of a cooling function allows more rapid temperature control.

### EXAMPLES

### Example 1

A temperature control test was performed using an acrylic resin microchip (8.5 cm×5 cm, thickness 1 mm). K-type thermocouples were used as the temperature sensors and fixed to the center of the microchip top surface. The heaters were of the transparent conductive film type. The temperature controller was of the PID control type. Temperature measurements were taken with K-type thermocouples and recorded on a notebook computer.

When the preset temperature was raised from 48 to 50° C. in 1 degree increments in the test, the temperature distribution inside the microchip remained within ±2.5° C. of the preset temperature in each case. The same test was also performed with aluminum foil (0.1 mm thick) of the same size as the microchip inserted between the microchip and heaters to further reduce the temperature distribution of the microchip. As a result, the temperature distribution of the microchip was within ±1° C. of the preset temperature at each temperature level.

### Example 2

Two kinds of DNA with different nucleotide sequences are separated using a DGGE microchip with the flow shown in FIG. 3.

PCR products of the V3 regions of 16s rRNA genes obtains from two different kinds of Sphingomonas are used as the DNA samples. In the preparation of the DNA samples, the two different microorganisms are first cultured in liquid medium, and collected by centrifugation. The cells are mixed, and DNA is extracted from the mixture by the benzyl chloride method. This extracted DNA is subjected to PCR using universal primers targeting the V3 region of the 16S rRNA gene (forward: 5′-CGCCCGCCGC GCGCGGCGGG CGGGGCGGGG GCACGGGGGG CCTACGGGAG GCAGCAG-3′ (SEQ ID NO 1); reverse: 5′-ATTACCGCGG CTGCTGG-3′ (SEQ ID NO 2)), and the resulting PCR product is the final DNA sample. The forward primer is provided with a GC clamp.

A microchip having a microchannel 100 μm wide and 25 μm deep formed by photolithography on Pyrex™ glass (7 cm×3.5 cm) is used in the test. This microchip is set on an inverted fluorescence microscope and detected with a photomultiplier tube. Urea and formamide are used as denaturants with a concentration gradient of 35 to 65%. Hydroxyethylcellulose (number-average molecular weight 90,000 to 105,000) is used as the DNA separation medium, and included in the electrophoresis buffer at a concentration of 1.5% (w/v). YOYO-1 is used as the DNA dye.

To separate the DNA, first the DNA sample is introduced into the separation microchannel with the temperature of the whole microchip controlled at the sample introduction temperature (30° C.). Next, DNA separation is initiated with the temperature controlled at the separation temperature (60° C.). After a fixed time, the temperature is controlled at the detection temperature (30° C.), and the peaks are detected. The temperature control system is the same one used in Example 1, and the temperature is controlled to within ±1° C. of each preset temperature.

As a result of the test, two peaks corresponding to the two microorganisms are detected. The two peaks are stably separated, and when the reproducibility of detection time is measured, the peaks are detected in roughly the same amount of time.

### Example 3

A system was constructed for independently controlling the temperature of one part of a microchip in order to spatially control the temperatures of the regions for performing the sample introduction process, separation process and detection process. A microchip (8.5 cm×5 cm, thickness 1 mm), copper plate (1 cm×4 cm, thickness 3 mm), Peltier element (8 mm×8 mm), copper plate and heat sink were affixed together in that order, and a thermistor was attached as the heat sensor to the copper plate contacting the microchip. The Peltier element was connected to a fixed voltage power source, and the output was controlled with a temperature controller. The temperature behavior of the copper plate attached to the microchip was measured using a K-type thermocouple as the preset temperature was varied. As a result, the variation in temperature measurements over time was within ±0.6° C. when the preset temperature was 30° C., 40° C., 50° C. and 60° C.

### Example 4

The temperatures for the sample introduction process, separation process and detection process were controlled over time.

The sample introduction and separation processes were controlled at 50° C., while after the separation process the temperature was lowered to 30° C. and DNA was detected at a fixed temperature of 30° C. in the detection process. A test was also performed with the temperature fixed at 50° C. in the sample introduction, separation and detection processes, and detection sensitivity was compared.

An acrylic resin microchip was used in the test. This microchip was set in an inverted fluorescence microscope, and detected with a photoelectric multiplier tube. Urea and formamide were used as denaturants, and the denaturant concentration was a uniform 60% throughout the test. Hydroxyethylcellulose (number-average molecular weight 90,000 to 105,000) was used as the DNA separation medium and included in the electrophoresis buffer at a concentration of 1.5% (w/v). YOYO-1 was used as the DNA dye. The PCR product of the V3 region of a 16S rRNA gene obtained from one kind of microorganism in the Sphingomonas genus was used as the DNA sample. Comparing the detected peaks, the peak area was 100 times larger when the detection process was at 30° C. than when it was at 50° C. This shows the importance for detection sensitivity of lowering the temperature in the detection process.

### Comparative Example 1

A comparative test with Example 1 was performed. The thermocouples were fixed to the transparent thin-film heaters rather than to the microchip. Temperature control was tested using this system.

As in Example 1, the preset temperature was raised from 48 to 50° C. in 1 degree increments. When the upper surface of the center of the microchips was measured with a thermocouple in this case, it differed by 5° C. or more from the preset temperature. When the temperature of the upper surface of the right side of the microchip was measured with a thermocouple, it differed by about 2° C. from the temperature of the upper surface of the center.

### Comparative Example 2

A comparative test with Example 2 was performed. The same DNA sample, microchip, detection device, DNA separation medium and electrophoresis buffer are used. The denaturant concentration is the same. The temperature control system is the one used in Comparative Example 1, and the test is performed under fixed temperature conditions of 60° C.

The two peaks are either not separated as a result of the test, or if they are separated there is variation in detection time, so reproducibility is poor in comparison with Example 2.

1. A microchip electrophoresis method for separating double-stranded nucleic acids by means of differences in nucleotide sequence while maintaining a preset temperature, wherein the temperature during the step of separating double-stranded nucleic acids is controlled within ±2.50° C. of said preset temperature.

2. The method according to claim 1, wherein at least one temperature selected from the temperature during the step of introducing double-stranded nucleic acids into an separation microchannel, the temperature during the step of separating double-stranded nucleic acids in the separation microchannel and the temperature during the step of detecting the separated double-stranded nucleic acids is controlled independently.

3. A microchip electrophoresis device for separating double-stranded nucleic acids inside an separation microchannel by means of differences in nucleotide sequence while maintaining a preset temperature, the microchip electrophoresis device comprising a temperature control device capable of controlling the temperature of the region of said separation microchannel within ±2.5° C. of said preset temperature.

4. The microchip electrophoresis device according to claim 3, further comprising a temperature control device capable of independently controlling at least one temperature selected from the temperature of the region of a sample introduction microchannel for introducing double-stranded nucleic acids into the separation microchannel, the temperature of the region of said separation microchannel and the temperature of the region for detecting the separated double-stranded nucleic acids.

5. The microchip electrophoresis device according to claim 3 or 4, wherein the microchip body is provided with one or a plurality of temperature sensors.

6. The microchip electrophoresis device according to claim 4, further comprising a plurality of heaters.
